(19) United States
(12) Patent Application Publication
(10) Pub. No.: US 2004/0018520 A1
(43) Pub. Date: Jan. 29, 2004

(54) Trans-splicing enzymatic nucleic acid mediated biopharmaceutical and protein

(76) Inventor: James Thompson, Lafayette, CO (US)

Correspondence Address: Anita J. Terpstra, McDonnell Boehnen Hulbert & Berghoff, 32nd Floor, 300 S. Wacker Drive, Chicago, IL, 60606, US

(21) Appl. No.: 10/421,015

(22) Filed: Apr. 22, 2003

Related U.S. Application Data
(60) Provisional application No. 60/374,427, filed on Apr. 22, 2002.

(51) Int. Cl. C12Q 1/68; C07H 21/04; A61K 48/00; C12P 19/34

(52) U.S. Cl. 435/6; 435/91.2; 514/44; 536/23.2

(57) ABSTRACT

The present invention relates to trans-splicing enzymatic nucleic acid molecules that are used to reprogram target genes and/or gene transcripts to express compounds within a cell, such as biopharmaceuticals, therapeutic proteins, tags, and reporters, useful in the treatment and diagnosis of diseases, illnesses, and/or related conditions.

This application claims the benefit of James Thompson et al., U.S. Provisional Application Serial No. 60/374,427, filed Apr. 22, 2002, which application is hereby incorporated by reference herein in its entirety, including the drawings.

### BACKGROUND OF THE INVENTION

The present invention relates to enzymatic nucleic acid mediated gene reprogramming and in vivo biopharmaceutical production applications thereof. The invention specifically provides compositions and methods for reprogramming endogenous genes to express trans-spliced sequences within cells. These sequences can encode biopharmaceutical compounds that are expressed in target cells, therefore providing therapeutic value to the cells or host organism containing such cells.

The following is a brief description of RNA splicing and RNA processing reactions. This summary is not meant to be complete but is provided only for understanding of the invention that follows. This summary is not an admission that the work described below is prior art to the claimed invention.

Prior to the 1970s it was thought that all genes were direct linear representations of the proteins that they encoded. This simplistic view implied that all genes were like ticker tape messages, with each triplet of DNA “letters” representing one protein “word” in the translation.

Protein synthesis occurred by first transcribing a gene from DNA into RNA (letter for letter) and then translating the RNA into protein (three letters at a time). In the mid 1970s it was discovered that some genes were not exact, linear representations of the proteins that they encode. These genes were found to contain interruptions in the coding sequence which were removed from, or “spliced out” of, the RNA before it became translated into protein. These interruptions in the coding sequence were given the name of intervening sequences (or introns) and the process of removing them from the RNA was termed splicing. A general reference for spliceosomes and how they are related to self-splicing introns is Guthrie, C., 1991, Science, 253, 157. After the discovery of introns, two questions immediately arose: (i) why are introns present in genes in the first place, and (ii) how do they get removed from the RNA prior to protein synthesis? The first question is still being debated, with no clear answer yet available. The second question, how introns get removed from the RNA, is much better understood after a decade and a half of intense research on this question. At least three different mechanisms have been discovered for removing introns from RNA. Two of these splicing mechanisms involve the binding of multiple protein factors which then act to correctly cut and join the RNA. A third mechanism involves cutting and joining of the RNA by the intron itself, in what was the first discovery of catalytic RNA molecules.

Cech and colleagues were trying to understand how RNA splicing was accomplished in a single-celled pond organism called Tetrahymena thermophila. They had chosen Tetrahymena thermophila as a matter of convenience, since each individual cell contains over 10,000 copies of one intron-containing gene (the gene for ribosomal RNA). They reasoned that such a large number of intron-containing RNA molecules would require a large amount of (protein) splicing factors to get the introns removed quickly. Their goal was to purify these hypothesized splicing factors and to demonstrate that the purified factors could splice the intron-containing RNA in vitro. Cech rapidly succeeded in achieving RNA splicing in vitro. As expected, splicing occurred when the intron-containing RNA was mixed with protein-containing extracts from Tetrahymena, but splicing also occurred when the protein extracts were left out. Cech proved that the intervening sequence RNA was acting as its own splicing factor to snip itself out of the surrounding RNA. They published this startling discovery in 1982. Continuing studies in the early 1980's served to elucidate the complicated structure of the Tetrahymena intron and to decipher the mechanism by which self-splicing occurs. Many research groups helped to demonstrate that the specific folding of the Tetrahymena intron is critical for bringing together the parts of the RNA that will be cut and spliced. Even after splicing is complete, the released intron maintains its catalytic structure. As a consequence, the released intron is capable of carrying out additional cleavage and splicing reactions on itself (to form intron circles). By 1986, Cech was able to show that a shortened form of the Tetrahymena intron could carry out a variety of cutting and joining reactions on other pieces of RNA. The demonstration proved that the Tetrahymena intron can act as a true enzyme: (i) each intron molecule was able to cut many substrate molecules while the intron molecule remained unchanged, and (ii) reactions were specific for RNA molecules that contained a unique sequence (CUCU) which allowed the intron to recognize and bind the RNA. Zaug and Cech coined the term “ribozyme” to describe any ribonucleic acid molecule that has enzyme-like properties. Also in 1986, Cech showed that the RNA substrate sequence recognized by the Tetrahymena ribozyme could be changed by altering a sequence within the ribozyme itself. This property has led to the development of a number of site-specific ribozymes that have been individually designed to cleave at other RNA sequences. The Tetrahymena intron is the most well-studied of what is now recognized as a large class of introns, Group I introns. The overall folded structure, including several sequence elements, is conserved among the Group I introns, as is the general mechanism of splicing. Like the Tetrahymena intron, some members of this class are catalytic, i.e., the intron itself is capable of the self-splicing reaction. Other Group I introns require additional (protein) factors, presumably to help the intron fold into and/or maintain its active structure. While the Tetrahymena intron is relatively large, (413 nucleotides) a shortened form of at least one other catalytic intron (SunY intron of phage T4, 180 nucleotides) may prove advantageous not only because of its smaller size but because it undergoes self-splicing at an even faster rate than the Tetrahymena intron.

Ribonuclease P (RNAseP) is an enzyme comprised of both RNA and protein components which are responsible for converting precursor tRNA molecules into their final form by trimming extra RNA off one of their ends. RNAseP activity has been found in all organisms tested, but the bacterial enzymes have been the most studied. The function of RNAseP has been studied since the mid-1970s by many labs. In the late 1970s, Sidney Altman and his colleagues showed that the RNA component of RNAseP is essential for its processing activity; however, they also showed that the protein component also was required for processing under their experimental conditions. After Cech's discovery of self-splicing by the Tetrahymena intron, the requirement for both protein and RNA components in RNAseP was reexamined. In 1983, Altman and Pace showed that the RNA was the enzymatic component of the RNAseP complex. This demonstrated that an RNA molecule was capable of acting as a true enzyme, processing numerous tRNA molecules without itself undergoing any change. The folded structure of RNAseP RNA has been determined, and while the sequence is not strictly conserved between RNAs from different organisms, this higher order structure is. It is thought that the protein component of the RNAseP complex may serve to stabilize the folded RNA in vivo. At least one RNA position important both to substrate recognition and to determination of the cleavage site has been identified, however little else is known about the active site. Because tRNA sequence recognition is minimal, it is clear that some aspect(s) of the tRNA structure must also be involved in substrate recognition and cleavage activity. The size of RNAseP RNA (>350 nucleotides), and the complexity of the substrate recognition, may limit the potential for the use of an RNAseP-like RNA in therapeutics. However, the size of RNAseP is being trimmed down (a molecule of only 290 nucleotides functions reasonably well). In addition, substrate recognition has been simplified by the recent discovery that RNAseP RNA can cleave small RNAs lacking the natural tRNA secondary structure if an additional RNA (containing a “guide” sequence and a sequence element naturally present at the end of all tRNAs) is present as well.

Symons and colleagues identified two examples of a self-cleaving RNA that differed from other forms of catalytic RNA already reported. Symons was studying the propagation of the avocado sunblotch viroid (ASV), an RNA virus that infects avocado plants. Symons demonstrated that as little as 55 nucleotides of the ASV RNA was capable of folding in such a way as to cut itself into two pieces. It is thought that in vivo self-cleavage of these RNAs is responsible for cutting the RNA into single genome-length pieces during viral propagation. Symons discovered that variations on the minimal catalytic sequence from ASV could be found in a number of other plant pathogenic RNAs as well. Comparison of these sequences revealed a common structural design consisting of three stems and loops connected by central loop containing many conserved (invariant from one RNA to the next) nucleotides. The predicted secondary structure for this catalytic RNA reminded the researchers of the head of a hammer; thus it was named as such. Ublenbeck was successful in separating the catalytic region of the ribozyme from that of the substrate. Thus, it became possible to assemble a hammerhead ribozyme from 2 (or 3) small synthetic RNAs. A 19-nucleotide catalytic region and a 24-nucleotide substrate were sufficient to support specific cleavage. The catalytic domain of numerous hammerhead ribozymes have now been studied by both the Uhlenbeck and Symons groups with regard to defining the nucleotides required for specific assembly and catalytic activity and determining the rates of cleavage under various conditions.

Haseloff and Gerlach showed it was possible to divide the domains of the hammerhead ribozyme in a different manner. By doing so, they placed most of the required sequences in the strand that didn't get cut (the ribozyme) and only a required UH where H=C, A, or U in the strand that did get cut (the substrate). This resulted in a catalytic ribozyme that could be designed to cleave any UH RNA sequence embedded within a longer “substrate recognition” sequence. The specific cleavage of a long mRNA, in a predictable manner using several such hammerhead ribozymes, was reported in 1988.

One plant pathogen RNA (from the negative strand of the tobacco ringspot virus) undergoes self-cleavage but cannot be folded into the consensus hammerhead structure described above. Bruening and colleagues have independently identified a 50-nucleotide catalytic domain for this RNA. In 1990, Hampel and Tritz succeeded in dividing the catalytic domain into two parts that could act as substrate and ribozyme in a multiple-turnover, cutting reaction. As with the hammerhead ribozyme, the hairpin catalytic portion contains most of the sequences required for catalytic activity while only a short sequence (GUC in this case) is required in the target. Hampel and Tritz described the folded structure of this RNA as consisting of a single hairpin and coined the term “hairpin” ribozyme (Bruening and colleagues use the term “paper clip” for this ribozyme motif). Continuing experiments suggest an increasing number of similarities between the hairpin and hammerhead ribozymes in respect to both binding of target RNA and mechanism of cleavage. At the same time, the minimal size of the hairpin ribozyme is still 50-60% larger than the minimal hammerhead ribozyme.

Hepatitis Delta Virus (HDV) is a virus whose genome consists of single-stranded RNA. A small region (about 80 nucleotides) in both the genomic RNA, and in the complementary anti-genomic RNA, is sufficient to support self-cleavage. As the most recently discovered ribozyme, HDV's ability to self-cleave has only been studied for a few years, but is interesting because of its connection to a human disease. In 1991, Been and Perrotta proposed a secondary structure for the HDV RNAs that is conserved between the genomic and anti-genomic RNAs and is necessary for catalytic activity. Separation of the HDV RNA into “ribozyme” and “substrate” portions has recently been achieved by Been, but the rules for targeting different substrate RNAs have not yet been determined fully. Been has also succeeded in reducing the size of the HDV ribozyme to .about.60 nucleotides.

A trans-splicing ribozyme can be employed to revise the sequence of targeted RNAs. A trans-splicing group I ribozyme from Tetrahymena has been used to repair truncated lacZ transcripts (Sullenger et al., 1994, Nature 371, 619; Sullenger et al., U.S. Pat. No. 5,667,969; incorporated by reference herein). In this system, a 3′ exon sequence encoding the restorative lacZ sequence was attached to the splicing ribozyme. For trans-splicing to correct the defective lacZ messages, the ribozyme recognizes the truncated 5′ lacZ transcript by base pairing, cleaves off additional nucleotides, retains the resulting 5′ lacZ cleavage product, and ligates the restorative lacZ 3′ exon sequence onto the cleaved 5′ product to yield the proper open reading frame for translation. The ribozyme in this example was shown to faithfully accomplish such RNA revision both in vitro and in Escherchia coli. Furthermore, in E. coli, the repaired RNAs were subsequently translated to produce a functional enzyme (Sullenger et al., supra).

Inoue et al., 1985, Cell 43, 431 states that short oligonucleotides of 2-6 nucleotides can undergo intermolecular exon ligation or splicing in trans. Inoue et al., also indicate that long 5′ exons should be reactive provided that three conditions are met: (1) the exon must have a 3′ hydroxyl group; (2) it must terminate in a sequence similar to that of the 3′ end of the 5′ exon; and (3) the 3′ terminal sequence must be available as opposed to being tied up in some secondary structure. Inoue et al. concludes that exon switching is possible, although limited by the availability of alternative 5′ exons that meet the above criteria. Further, these include transcripts that are not 5′ exons from other precursors, since RNA polymerases always leave 3′ hydroxyl ends.

Haseloff et al., U.S. Pat. Nos. 6,071,730; 6,010,904; 5,882,907; 5,866,384; 5,863,774; 5,849,548; and 5,641,673 describes a method of cell ablation using diphtheria toxin expressed from cells treated with trans-splicing ribozymes.

Haseloff et al., U.S. Pat. Nos. 6,015,794 and 5,874,414, describes trans-splicing ribozymes based upon the catalytic core of a Group I intron.

Mitchell et al., U.S. Pat. Nos. 6,280,978 and 6,083,702, describes a method of spliceosome mediated trans-splicing that can be used to selectively kill target cells.

### SUMMARY OF THE INVENTION

The present invention features methods and compositions for reprogramming genes. Specifically, the instant invention features enzymatic nucleic acid molecules that are designed to interact with endogenous nucleic acid molecules (DNA and/or RNA) within a cell and mediate trans-splicing or reverse trans-slicing reactions resulting in the generation of nucleic acid molecules having sequence encoding a biopharmaceutical composition. Cells treated with enzymatic nucleic acid molecules of the invention can be used to express biopharmaceutical compositions useful in treating disease and illness. The expression of biopharmaceutical compositions can thus be regulated by the genetic control of the parental gene target within target cell(s) in response to specific conditions and/or factors that regulate gene expression that are specific to the target cell type chosen for reprogramming.

In one embodiment, the present invention provides methods and compositions for generating biopharmaceutical molecules through targeted enzymatic nucleic acid mediated trans-splicing. The compositions of the invention include enzymatic nucleic acid molecules designed to interact with a natural target RNA molecule and mediate a trans-splicing reaction resulting in the formation of a novel chimeric RNA molecule. The enzymatic nucleic acid molecules of the invention are genetically engineered so as to result in the production of a novel chimeric RNA that encodes a biopharmaceutical protein. In another embodiment, the target RNA is chosen as a target because it is expressed within a specific cell type that provides a means for targeting expression of the novel chimeric RNA to a selected cell type.

In one embodiment, the invention features enzymatic nucleic acid molecules that have been genetically engineered for the identification of exon/intron boundaries of pre-mRNA molecules using an exon tagging method. In another embodiment, enzymatic nucleic acid molecules of the invention can be designed to produce chimeric RNA molecules encoding peptide affinity purification tags which can be used to purify and identify proteins expressed in a specific cell type.

In one embodiment, the invention features an enzymatic nucleic acid molecule having trans-splicing activity, wherein the enzymatic nucleic acid molecule comprises sequence encoding a biopharmaceutical. The biopharmaceutical encoding sequence can comprise RNA or DNA, such that trans-splicing activity of the enzymatic nucleic acid molecule results in the expression of the biopharmaceutical in vitro or in vivo. In one embodiment, a biopharmaceutical composition of the invention comprises a compound featured in Table I.

In one embodiment, the invention features a method comprising: (a) providing an enzymatic nucleic acid molecule of the invention and a substrate comprising a predetermined target RNA for the enzymatic nucleic acid molecule in vitro under reaction conditions that promote trans-splicing activity of the enzymatic nucleic acid molecule; and (b) reacting the enzymatic nucleic acid molecule with the substrate.

In one embodiment, the invention features a method comprising: (a) providing an enzymatic nucleic acid molecule of the invention and a substrate comprising a predetermined target DNA for the enzymatic nucleic acid molecule in vitro under reaction conditions that promote trans-splicing activity of the enzymatic nucleic acid molecule; and (b) reacting the enzymatic nucleic acid molecule with the substrate.

In one embodiment, the invention features a method comprising introducing into a cell an enzymatic nucleic acid molecule of the invention, wherein the enzymatic nucleic acid molecule comprises sequence encoding a biopharmaceutical, and wherein the enzymatic nucleic acid molecule further comprises binding region(s) complementary to a target RNA sequence in the cell, under conditions suitable for trans-splicing activity such that the sequence encoding a biopharmaceutical is introduced into the target RNA sequence in a manner that allows the biopharmaceutical to be expressed under the genetic control of the host cell. In a non-limiting example, the target RNA molecule can be a messenger RNA (mRNA), pre-messenger RNA (pre-mRNA), a structural/functional RNA (e.g., a tRNA, rRNA or 7 SLRNA), or viral RNA.

In one embodiment, the invention features a method comprising introducing into a cell an enzymatic nucleic acid molecule of the invention, wherein the enzymatic nucleic acid molecule comprises sequence encoding a biopharmaceutical, and wherein the enzymatic nucleic acid molecule further comprises binding region(s) complementary to a target DNA sequence in the cell, under conditions suitable for reverse trans-splicing activity such that the sequence encoding a biopharmaceutical is introduced into the target DNA sequence in a manner that allows the biopharmaceutical to be expressed under the genetic control of the host cell. In a non-limiting example, the target DNA is chromosomal DNA or viral DNA.

In one embodiment, the invention features an RNA or DNA expression vector, wherein the vector is capable of being stably maintained in a host or inserted into the genome of a host, and wherein the vector provides a promoter sequence capable of functioning in such host and which is operably linked to the sequence of an enzymatic nucleic acid molecule of the invention.

In another embodiment, the invention features a method comprising: (a) introducing into a cell an expression vector of the invention encoding an enzymatic nucleic acid molecule, wherein the enzymatic nucleic acid molecule comprises sequence encoding a biopharmaceutical, and wherein the enzymatic nucleic acid molecule further comprises binding region(s) complementary to a target RNA sequence in the cell, under conditions suitable for expression of the enzymatic nucleic acid molecule in the cell; and (b) reprogramming the expression of the target RNA via trans-splicing activity with the enzymatic nucleic acid molecule by introducing the sequence encoding a biopharmaceutical into the target RNA sequence under conditions suitable for the biopharmaceutical to expressed in the cell. In a non-limiting example, the target RNA molecule can be a messenger RNA (mRNA), pre-messenger RNA (pre-mRNA), structural/functional RNA or viral RNA.

In another embodiment, the invention features a method comprising: (a) introducing into a cell an expression vector of the invention encoding an enzymatic nucleic acid molecule, wherein the enzymatic nucleic acid molecule comprises sequence encoding a biopharmaceutical, and wherein the enzymatic nucleic acid molecule further comprises binding region(s) complementary to a target DNA sequence in the cell, under conditions suitable for expression of the enzymatic nucleic acid molecule in the cell; and (b) reprogramming the expression of the target DNA via reverse trans-splicing activity with the enzymatic nucleic acid molecule by introducing the sequence encoding a biopharmaceutical into the target DNA sequence under conditions suitable for the biopharmaceutical to expressed in the cell. In a non-limiting example, the target DNA is chromosomal DNA or viral DNA.

In one embodiment, the invention features a method for generating enzymatic nucleic acid molecules with trans-splicing activity capable of biopharmaceutical production comprising: (a) generating a randomized pool of oligonucleotides, wherein a portion of each oligonucleotide comprises a fixed sequence that encodes a biopharmaceutical product and wherein a portion of each oligonucleotide comprises sequence complementary to a predetermined nucleic acid target sequence; (b) isolating sequences from the pool that possess trans-splicing or reverse trans-splicing activity; (c) amplifying the sequences isolated from (b) under conditions suitable for introducing some degree of mutation into the sequences; and (d) repeating steps (b) and (c) under conditions suitable for isolating enzymatic nucleic acid molecules with trans-splicing activity capable of introducing the sequence encoding a biopharmaceutical product into the nucleic acid target sequence. The enzymatic nucleic acid molecules generated by the method of the invention can support biopharmaceutical production in vitro or in vivo. The predetermined nucleic acid target sequence in the method of the invention can comprise RNA or DNA, for example, messenger RNA (mRNA), pre-messenger RNA (pre-mRNA), viral RNA, chromosomal DNA or viral DNA.

Essentially any nucleic acid sequence encoding a protein can be introduced into any target DNA or RNA sequence using the methods of the instant invention, thus providing novel enzymatic nucleic acid trans-splicing molecules that can be specific for a particular biopharmaceutical compound or that can alternately be adapted for use with different biopharmaceutical compounds using additional evolutionary selection schemes or rational design approaches. In addition, these novel constructs can be chemically modified as described herein to modify various properties of the construct including, but not limited to, catalytic activity, bioavailability and/or nuclease resistance.

In another embodiment, the methods of the invention are used to express biopharmaceutical tags or reporters within cells. Such tags and reporters can be used to visualize cells and/or tissues comprising such cells that express the biopharmaceutical tag or reporter. In a non-limiting example, the compositions and methods of the invention are used to reprogram a nucleic acid sequence encoding a gene that is expressed in cancerous cells to express green fluorescent protein (GFP) or an equivalent protein that allows the cancerous cells to be distinguished from non-cancerous cells. Reprogramming in this manner is useful in a variety of applications, including use in biopsies and surgeries to detect and/or remove cancerous tissue or in detecting the presence of cancerous lesions, for example using a CAT scan or MRI. In surgical applications, the expression of the biopharmaceutical tag or reporter will allow more accurate excision of cancerous tissue by allowing the practitioner to determine if a given procedure has removed all cancerous tissue from a patient. As such, excessive removal of tissues surrounding a tumor can be avoided, thereby better preserving the normal function of the affected area. This methodology would be especially useful, for example, in brain surgery to remove a brain tumor. The target nucleic acid sequence this is reprogrammed can be a gene or gene transcript that is overexpressed or is exclusively expressed in the cells and tissues of interest, such that expression of the biopharmaceutical tag or reporter can be sufficiently distinguished from normal cells.

In one embodiment, an enzymatic nucleic acid trans-splicing molecule of the invention comprises about 8 to about 100 bases complementary to the target nucleic acid (DNA or RNA). In another embodiment, an enzymatic nucleic acid trans-splicing molecule of the invention comprises about 14 to about 24 bases complementary to the target nucleic acid (DNA or RNA).

In another embodiment, an enzymatic nucleic acid trans-splicing molecule of the invention comprises at least one 2′-sugar modification.

In another embodiment, an enzymatic nucleic acid trans-splicing molecule of the invention comprises at least one nucleic acid base modification.

In another embodiment, an enzymatic nucleic acid trans-splicing molecule of the invention comprises at least one phosphate backbone modification.

In one embodiment, the invention features a mammalian cell, for example a human cell, including the enzymatic nucleic acid trans-splicing molecule of the invention.

In one embodiment, the invention features a method of expressing a biopharmaceutical composition in a cell comprising contacting the cell with an enzymatic nucleic acid trans-splicing molecule of the invention under conditions suitable for the expression.

In one embodiment, the invention features a method of treating a patient having a disease, illness, or condition that can be treated with a biopharmaceutical compound, comprising contacting cells of the patient with an enzymatic nucleic acid trans-splicing molecule of the invention under conditions suitable for the treatment.

In another embodiment, a method of treatment further comprises the use of one or more drug therapies under conditions suitable for the treatment.

In another embodiment, the invention features a method of trans-splicing a sequence encoding a biopharmaceutical compound into an endogenous DNA or RNA sequence, comprising contacting an enzymatic nucleic acid trans-splicing molecule of the invention with a target DNA or RNA molecule under conditions suitable for the sequence to be trans-spliced into the target DNA or RNA sequence.

In one embodiment, the enzymatic nucleic acid trans-splicing molecules of the invention are derived from group I introns (Sullenger et al., supra) or group II introns (Jacquier, 1990, TIBS 15, 351; Michels et al., 1995, Biochemistry, 34, 2965; Chanfreau et al., 1994, Science, 266, 1383; Mueller et al., 1993, Science, 261, 1035; Jarrell et al., U.S. Pat. No. 5,498,531).

In another embodiment, the enzymatic nucleic acid trans-splicing molecules of the invention facilitate a trans-splicing reaction in the presence of one or more cellular factors, such as protein factors (Bruzik et al., supra; Jarrell supra; Ghetti et al., 1995, Proc. Natl. Acad. Sci., 92, 11461). In yet another embodiment, the enzymatic nucleic acid trans-splicing molecules are derived from pre-messenger RNA introns, but can also be derived from other introns such as group I and group II introns.

In one embodiment, an enzymatic nucleic acid trans-splicing molecule of the invention comprises a cap structure, for example, a 3′,3′-linked or 5′,5′-linked deoxyabasic ribose derivative, wherein the cap structure is at the 5′-end, or 3′-end, or both the 5′-end and the 3′-end of the enzymatic nucleic acid molecule.

In one embodiment, the invention features an expression vector comprising a nucleic acid sequence encoding at least one enzymatic nucleic acid trans-splicing molecule of the invention, in a manner which allows expression of the enzymatic nucleic acid trans-splicing molecule. In another embodiment, the invention features a mammalian cell, for example, a human cell, including an expression vector of the invention.

In one embodiment, an expression vector of the invention comprises a nucleic acid sequence encoding two or more enzymatic nucleic acid trans-splicing molecules, which can be the same or different.

In another embodiment, the invention features a method for the treatment of a variety pathologic indications, including cancer, infectious disease, cardiovascular, neurologic, inflammatory, immunologic, metabolic, endocrine, or genetic diseases and disorders or any other disease or condition that can be treated with a biopharmaceutical composition in a patient, comprising administering to the patient an enzymatic nucleic acid trans-splicing molecule of the invention under conditions suitable for the treatment, including administering to the patient one or more other therapies.

In another embodiment, the invention features a method of administering to a mammal, for example a human, an enzymatic nucleic acid trans-splicing molecule of the invention comprising contacting the mammal with the enzymatic nucleic acid trans-splicing molecule under conditions suitable for the administration of the enzymatic nucleic acid, for example, in the presence of a delivery reagent such as a lipid, cationic lipid, phospholipid, or liposome.

In another embodiment, the invention features a method of administering to a mammal, for example a human, a vector encoding an enzymatic nucleic acid trans-splicing molecule of the invention comprising contacting the mammal with the vector under conditions suitable for the administration of the vector, for example, in the presence of a delivery reagent such as a lipid, cationic lipid, phospholipid, or liposome.

In another embodiment, the invention features a method of administering to a patient an enzymatic nucleic acid trans-splicing molecule of the invention in conjunction with a therapeutic agent comprising contacting the patient with the enzymatic nucleic acid trans-splicing molecule and the therapeutic agent under conditions suitable for the administration.

The term “enzymatic nucleic acid molecule”, or “enzymatic nucleic acid trans-splicing molecule” as used herein refers to a nucleic acid molecule that has complementarity in a substrate binding region to a specified genetic target, such as mRNA, pre-mRNA, viral RNA, chromosomal DNA, bacterial DNA, or viral DNA, and also has an enzymatic activity wherein a nucleic acid sequence of a first polynucleotide is co-linearly linked to or inserted co-linearly into the sequence of a second polynucleotide, in a manner that retains the 3′-5′ phosphodiester linkage between the polynucleotides. That is, the enzymatic nucleic acid trans-splicing molecule is able to reprogram the expression of a target nucleic acid. The complementary regions of the enzymatic nucleic acid trans-splicing molecule allow sufficient hybridization of the enzymatic nucleic acid molecule to the target nucleic acid molecule and thus permit trans-splicing. One hundred percent complementarity is preferred, but complementarity as low as 50-75% can also be useful in this invention. The enzymatic nucleic acid molecules can comprise RNA or DNA and can be modified at the nucleotide base, sugar, and/or phosphate groups. The specific enzymatic nucleic acid molecules described in the instant application are not limiting in the invention and those skilled in the art will recognize that all that is important in an enzymatic nucleic acid molecule of this invention is that it has a specific substrate binding site that is complementary to one or more of the target nucleic acid regions, and that it have nucleotide sequences within or surrounding that substrate binding site which impart a nucleic acid trans-splicing activity to the molecule.

The term “trans-splicing” as used herein refers to a form of genetic manipulation wherein a nucleic acid sequence of a first polynucleotide is co-linearly linked to or inserted co-linearly into the sequence of a second polynucleotide, in a manner that retains the 3′-5′ phosphodiester linkage between the polynucleotides. By “directed” trans-splicing or “substrate-specific” trans-splicing is meant a trans-splicing reaction that requires a specific species of RNA or DNA as a substrate for the trans-splicing reaction (that is, a specific species of RNA or DNA in which to splice the transposed sequence). Directed trans-splicing can target more than one RNA or DNA species if the enzymatic nucleic acid molecule is designed to be directed against a target sequence present in a related set of RNA or DNA sequences. The term “trans-splicing activity” refers to the co-linear linking or insertion of a first polynucleotide sequence into a second polynucleotide sequence, as well as the ability to effect such linking or insertion. The term “nucleic acid molecule” as used herein refers to a molecule having nucleotides.

The nucleic acid can be single, double, or multiple stranded and can comprise modified or unmodified nucleotides or non-nucleotides or various mixtures and combinations thereof.

The term “substrate binding region” or “substrate binding domain” as used herein refers to that portion/region of an enzymatic nucleic acid that is able to interact, for example via complementarity (i.e., able to base-pair with), with a portion of its substrate. Such complementarity can be 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). That is, these regions contain sequences within an enzymatic nucleic acid that are intended to bring the enzymatic nucleic acid and the target nucleic acid together through complementary base-pairing interactions. The enzymatic nucleic acid of the invention can have binding regions that are contiguous or non-contiguous and can be of varying lengths. The length of the binding region(s) can be greater than or equal to four nucleotides and of sufficient length to stably interact with a target nucleic acid; in one embodiment they can be 12-100 nucleotides; in another embodiment they can be 14-24 nucleotides long (see for example Werner and Uhlenbeck, supra; Hamman et al., supra; Hampel et al., EP0360257; Berzal-Herranze et al., 1993, EMBO J., 12, 2567-73) or 8-14 nucleotides long. If two binding regions are chosen, the design is such that the length of the binding regions are symmetrical (i.e., each of the binding regions is of the same length; e.g., four and four, five and five nucleotides, or six and six nucleotides, or seven and seven nucleotides long) or asymmetrical (i.e., the binding regions are of different length; e.g., three and five, six and three nucleotides; three and six nucleotides long; four and five nucleotides long; four and six nucleotides long; four and seven nucleotides long; and the like).

The term “randomized pool” as used herein refers to a group of oligonucleotides that contain regions of completely random sequence and/or partially random sequence. By completely random sequence is meant a sequence wherein theoretically there is equal representation of A, U, G and C nucleotides or modified derivatives thereof, at each position in the sequence. By partially random sequence is meant a sequence wherein there is an unequal representation of A, U, G and C nucleotides or modified derivatives thereof, at each position in the sequence. A partially random sequence can therefore have one or more positions of complete randomness and one or more positions with defined nucleotides.

The term “biopharmaceutical” or “biopharmaceutical compound” as used herein refers to any compound such as a protein, peptide, or polypeptide, which can be expressed endogenously in a biological system under genetic control and which confers biological activity toward pharmaceutical or therapeutic use. The biopharmaceutical or biopharmaceutical compound can be constitutively or inducibly expressed. The biological system can be an in vivo biological system and/or an in vitro biological system.

The term “biopharmaceutical tag” or biopharmaceutical reporter” as used herein refers to any compound such as a protein, peptide, or polypeptide, which can be expressed endogenously in a biological system under genetic control and which confers physical or chemical properties when expressed that can be used to distinguish or detect such expression in a biological system.

The term “biological system” as used herein can be a eukaryotic system or a prokaryotic system, for example a bacterial cell, plant cell or a mammalian cell, or of plant origin, mammalian origin, yeast origin, Drosophila origin, or archebacterial origin.

The term “sufficient length” as used herein refers to an oligonucleotide of greater than or equal to 3 nucleotides that is of a length great enough to provide the intended function under the expected condition. For example, for binding regions of enzymatic nucleic acid “sufficient length” means that the binding region sequence is long enough to provide stable binding to a target site under the expected binding conditions. The binding regions are not so long as to prevent useful turnover of the nucleic acid molecule.

The term “stably interact” as used herein refers to interaction of the enzymatic nucleic acid molecules of the invention with a target nucleic acid (e.g., by forming hydrogen bonds with complementary nucleotides in the target under physiological conditions) that is sufficient to the intended purpose (e.g., trans-splicing of target RNA).

The term “homology” as used herein is used in its usual biological sense, such that the nucleotide sequence of two or more nucleic acid molecules is partially or completely identical.

The term “gene” as used herein refers to a nucleic acid that encodes an RNA, for example, nucleic acid sequences including but not limited to structural genes encoding a polypeptide.

The term “complementarity” as used herein refers to the ability of a nucleic acid to form hydrogen bond(s) with another nucleic acid molecule by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid trans-splicing, cleavage, or ligation. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The term “RNA” as used herein refers to a molecule comprising at least one ribonucleotide residue. By “ribonucleotide” or “2″-OH” is meant a nucleotide with a hydroxyl group at the 2′ position of a β-D-ribo-furanose moiety.

The term “DNA” as used herein refers to a molecule comprising at least one deoxyribonucleotide residue. By “deoxyribonucleotide” is meant a nucleotide lacking a hydroxyl group at the 2′ position of a β-D-ribo-furanose moiety.

In one embodiment, the enzymatic nucleic acid trans-splicing molecules of the invention comprise about 20 to about 10000 nucleotides. In another embodiment, the enzymatic nucleic acid trans-splicing molecules of the invention comprise about 50 to about 5000 nucleotides. In yet another embodiment, the enzymatic nucleic acid trans-splicing molecules of the invention comprise about 100 to about 1000 nucleotides.

The invention provides a method for producing a class of nucleic acid-based gene modulating agents that exhibit a high degree of specificity for the RNA or DNA of a desired target nucleic acid sequence. For example, the enzymatic nucleic acid trans-splicing molecules of the invention can be targeted to a highly conserved sequence region of target nucleic acids that are chosen to be reprogrammed such that expression of biopharmaceutical compositions can be provided with either one or several nucleic acid molecules of the invention. Such nucleic acid molecules can be delivered exogenously to specific tissue or cellular targets as required. Alternatively, the nucleic acid molecules can be expressed from DNA and/or RNA vectors that are delivered to specific cells.

The term “cell” as used herein is used in its usual biological sense, and does not refer to an entire multicellular organism. The cell can, for example, be in vitro, e.g., in cell culture, or present in a multicellular organism, including, e.g., birds, plants and mammals such as humans, cows, sheep, apes, monkeys, swine, dogs, and cats. The cell may be prokaryotic (e.g., bacterial cell) or eukaryotic (e.g., mammalian or plant cell).

The term “highly conserved sequence region” as used herein refers to a nucleotide sequence of one or more regions in a target gene that does not vary significantly from one generation to the other or from one biological system to the other.

The enzymatic nucleic acid trans-splicing molecules of the invention that are used to reprogram gene expression are useful for generating biopharmaceutical compositions that are useful in the prevention and/or treatment of various diseases, illnesses or conditions, including but not limited to cancer, infectious disease, cardiovascular, neurologic, inflammatory, immunologic, metabolic, endocrine, or genetic diseases and disorders or any other diseases or conditions that can be treated with an biopharmaceutical composition in a cell or tissue, alone or in combination with other therapies.

The nucleic acid molecules of the invention can be added directly, or can be complexed with cationic lipids, packaged within liposomes, or otherwise delivered to target cells or tissues, for example by pulmonary delivery of an aerosol formulation with an inhaler or nebulizer. The nucleic acid or nucleic acid complexes can be locally administered to relevant tissues ex vivo, or in vivo through inhalation, injection or infusion pump, with or without their incorporation in biopolymers.

In another embodiment, the enzymatic nucleic acid trans-splicing molecules of the invention comprise nucleotide or non-nucleotide linkers. The term “non-nucleotide” as used herein includes either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000. A “non-nucleotide” linker further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to trans-splice a target RNA or DNA molecule.

In another embodiment, enzymatic nucleic acid trans-splicing molecules that interact with target nucleic acid molecules and have trans-splicing activity are expressed from transcription units inserted into DNA or RNA vectors. The recombinant vectors can be DNA plasmids or viral vectors. Nucleic acid molecule expressing viral vectors can be constructed based on, but not limited to, adeno-associated virus, retrovirus, adenovirus, or alphavirus. The recombinant vectors capable of expressing the nucleic acid molecules can be delivered as described above, and persist in target cells. Alternatively, viral vectors can be used that provide for transient expression of the nucleic acid molecules. Such vectors can be repeatedly administered as necessary. Once expressed, the nucleic acid molecules can bind to the target nucleic acid sequence and reprogram its function or expression, such as in expressing biopharmaceutical compositions. Delivery of nucleic acid molecule expressing vectors can be systemic, such as by intravenous or intramuscular administration, by administration to target cells ex-planted from the patient followed by reintroduction into the patient, or by any other means that would allow for introduction into the desired target cell. DNA-based nucleic acid molecules of the invention can be expressed via the use of a single stranded DNA intracellular expression vector or any other similar approach.

The term “vectors” as used herein refers to any nucleic acid- and/or viral-based technique used to deliver a desired nucleic acid.

The term “patient” or “subject” as used herein refers to an organism, which is a donor or recipient of explanted cells, or the cells themselves. “Patient” or “subject” also refers to an organism to which the nucleic acid molecules of the invention can be administered. A patient or subject can be a mammal or mammalian cells. In one embodiment, a patient or subject is a human or human cells.

The term “enhanced enzymatic activity” as used herein refers to include activity measured in cells and/or in vivo where the activity is a reflection of both the enzymatic or catalytic activity and the stability of the nucleic acid molecules of the invention. In this invention, the product of these properties can be increased in vivo compared to an all RNA enzymatic nucleic acid or all DNA enzyme. In some cases, the activity or stability of the nucleic acid molecule can be decreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition with nucleic acid molecules of the invention that reprogram nucleic acid sequences within cells to produce biopharmaceutical compositions, the patient can be treated, or other appropriate cells can be treated, as is evident to those skilled in the art, individually or in combination with one or more drugs under conditions suitable for the treatment.

In a further embodiment, the nucleic acid molecules of the invention can be used in combination with other known treatments to treat conditions or diseases discussed herein. For example, the described molecules can be used in combination with one or more known therapeutic agents to treat a given disease, illness, or condition that responds to treatment with biopharmaceutical compositions described herein.

Other features and advantages of the invention will be apparent from the following description of the preferred embodiments thereof, and from the claims.

### BRIEF DESCRIPTION OF THE DRAWINGS

FIG. 1 shows a scheme for generating biopharmaceuticals using targeted trans-splicing.

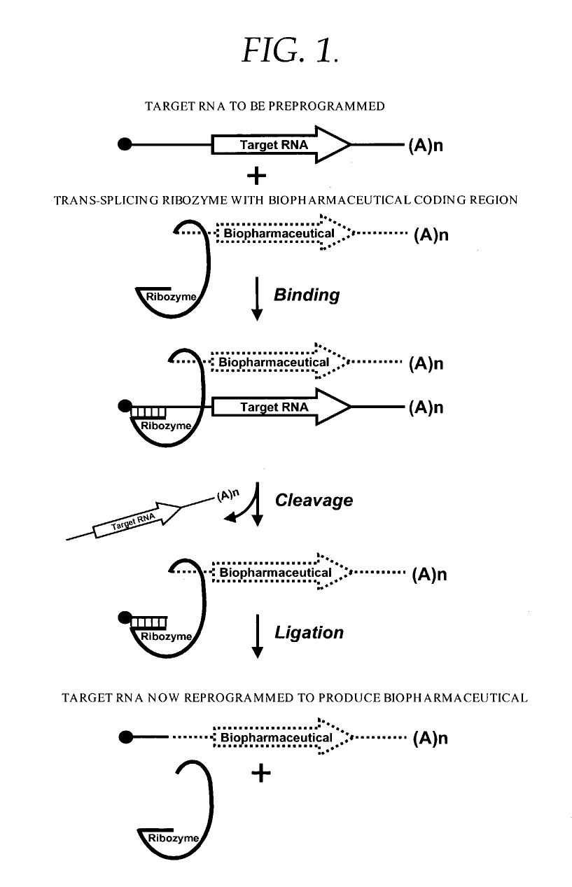

FIG. 2 shows a scheme for generating a biopharmaceutical tag or reporter using targeted trans splicing enzymatic nucleic acid molecule (ribozyme).

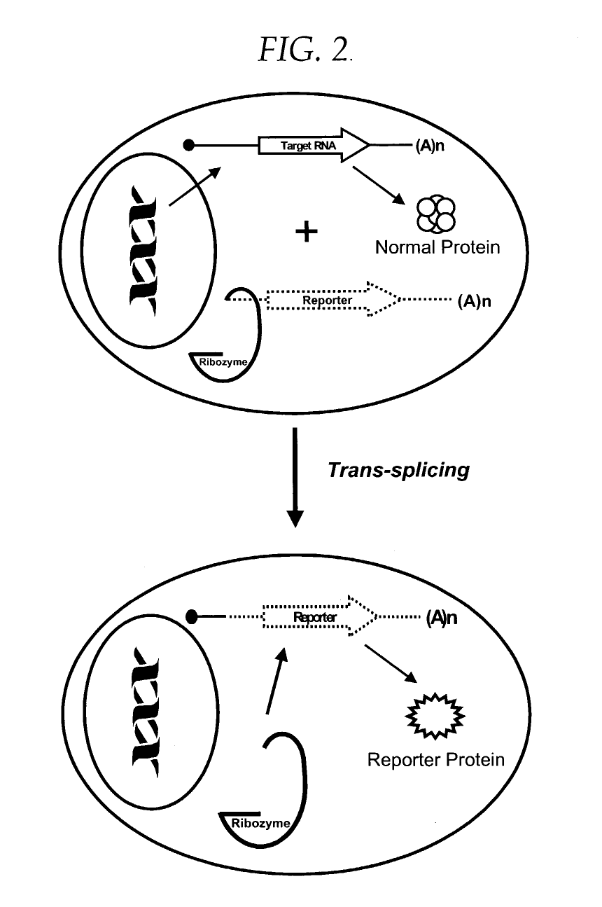

### DETAILED DESCRIPTION OF THE INVENTION

Nucleic Acid Engineering and Mechanism of Action

During gene expression, the information contained in a given protein encoding gene is directly copied into the corresponding pre-messenger RNA by transcription. The information embedded in this RNA is not fixed however and can be modified by splicing or editing to remove, add or rewrite parts of the initial transcript. The self-splicing reaction of the group I intron ribozyme from Tetrahymena thermophila is perhaps the most thoroughly understood reaction that revises RNA. The intron performs two consecutive transesterification reactions to liberate itself and join flanking exon sequences. Careful analysis of this self-splicing reaction has illustrated that the vast majority of sequence requirements for such excision are contained within the intron. No specific sequence requirements exist for the 3′ exon, and the only specific sequence requirement for 5′ exons is to have a uridine (U) preceding the cleavage site. For effective trans-splicing to take place, base pairing is maintained between the end of the 5′ exon and the 5′ exon-binding site present in the ribozyme such that the ribozyme can hold onto the 5′ exon after cleavage. These base pairs can be composed of any sequence of complementary nucleotides.

In addition to performing self-splicing, the group I ribozyme from Tetrahymena can trans-splice an exon attached to its 3′ end onto a separate 5′ exon RNA. In this reaction, the 5′ exon is not covalently attached to the ribozyme but is bound via base pairing through the 5′ exon binding site on the ribozyme. In the process of pairing, a U is positioned across from the guanosine present at the 5′ end of the 5′ exon binding site. Once positioned, the ribozyme cleaves the bound substrate RNA at the reconstructed 5′ splice site and ligates its 3′ exon onto the 5′ exon cleavage product. Trans-splicing by group I ribozymes is extremely malleable. Virtually any U residue in a 5′ exon can be targeted for splicing by altering the nucleotide composition of the 5′ exon binding site on the ribozyme to make it complementary to a target sequence present on the substrate RNA. Because no specific 3′ exon sequences are required, virtually any 3′ exon sequence can be spliced onto a targeted U residue by such a reaction.

As described herein, the directed trans-splicing enzymatic nucleic acid molecules of the invention can be engineered using the catalytic core if the group I intron or can be alternately designed using in vitro selection by methods of the invention. The group I intron, and its catalytic core can be isolated by methods known in the art. The catalytic core of the intron, that is, the truncated intron, differs from the full-length intron only in that it is truncated at the ScaI site, thus removing the last five nucleotides of the intron. The truncated intron RNA can be prepared by techniques known in the art. Transcribed Tet.1 cDNA may be used as the substrate for polymerase chain reaction (PCR) mutagenesis to produce a synthetic trans-splicing enzyme. In addition, chemically modified nucleotides can be introduced into the sequence of transcribed trans-splicing enzymatic nucleic acid molecules of the invention using chemically modified nucleoside triphosphates, for example 2′-deoxy-2′-fluro, 2′-deoxy-2′-amino, 2′-O-methyl or any other modified nucleoside triphosphate that can be enzymatically incorporated into a transcribed sequence.

Substrate specificity of the enzymatic nucleic acid molecules of the invention, that is, the ability of the trans-splicing nucleic acid molecule to “target” a specific RNA or DNA as a substrate, can be conferred by fusing complementary sequences specific to the target (substrate) nucleic acid to the 5′ terminus of the enzymatic nucleic acid molecule. Directed trans-splicing specificity of the nucleic acid molecules of the invention, that is, specificity in trans-splicing a desired exogenous sequence of interest with the sequence of a target nucleic acid (DNA or RNA), is conferred by providing a new 3′ exon at the 3′ terminus of the enzymatic nucleic acid molecule.

To alter the structural and catalytic properties of the Group I introns, exon sequences replace the flanking sequences of such introns so that only the catalytic core of the intron, the enzymatic nucleic acid, remains. The resulting modified enzymatic nucleic acid can interact with substrate nucleic acid sequences in trans. When truncated forms of the intron (i.e., the catalytic “core,” i.e. truncated at the ScaI site, removing the last five nucleotides of the intron) are incubated with sequences corresponding to the 5′ splice junction of the native enzymatic nucleic acid molecule, the site undergoes guanosine-dependent cleavage in mimicry of the first step in splicing. Generally, engineering of group I intron derived enzymatic nucleic acid molecules of the invention can follow the guidelines described in Haseloff et al., U.S. Pat. No. 6,015,794; and Sullenger et al., U.S. Pat. No. 5,869,254 and 5,667,969, all incorporated by reference herein.

Trans-splicing enzymatic nucleic acid molecules of the invention can be designed to effectively trans-splice essentially any nucleic acid sequence onto any nucleic acid target to reprogram the expression of the nucleic acid target sequence. The target sequence need not contain an intron sequence nor require that the enzymatic nucleic acid molecule be an intron in the target sequence. For example, a generalized strategy for such design can include: (a) the identification of the desired target nucleic acid sequence; (b) cloning and/or sequencing of the desired target nucleic acid sequence or portion thereof; (c) selection of a desired coding sequence to trans-splice into the target nucleic acid sequence; (d) the construction of a trans-splicing enzymatic nucleic acid molecule of the invention capable of hybridizing to such target using the guidelines described herein; (e) confirmation that the trans-splicing enzymatic nucleic acid molecule of the invention will utilize the target as a substrate for the specific trans-splicing reaction that is desired; and (f) the introduction of the trans-splicing enzymatic nucleic acid molecule into the desired cell.

Choice of a target nucleic acid sequence can reflect the desired purpose of the trans-splicing reaction. When the trans-splicing reaction is used to provide expression of a biopharmaceutical composition in a host cell, then the choice of the target nucleic acid sequence will reflect the desired expression pattern of the biopharmaceutical composition. If it is desired that the composition be continuously expressed by the host, then the target nucleic acid should also to be continuously expressed. If it is desired that the composition be selectively expressed only under a desired growth, developmental, hormonal, or environmental condition, then the target nucleic acid should also be selectively expressed under such conditions. For example, if it is desired that the composition be selectively expressed in a particular cell or tissue, then the target nucleic acid should be expressed in a cell or tissue-specific manner. It is not necessary that expression of the enzymatic nucleic acid molecule itself be selectively limited to a desired growth, developmental, hormonal, or environmental condition if the substrate for such enzymatic nucleic acid molecule is not otherwise present in the host as the enzymatic nucleic acid molecule itself is not translated by the host. Thus, sequences encoded by the nucleic acid sequence provided by the enzymatic nucleic acid molecule of the invention are not translated until the trans-splicing event occurs and such event can optionally be controlled by the expression of the enzymatic nucleic acid molecule substrate in the host.

If desired, expression of the enzymatic nucleic acid molecule can be engineered to occur in response to the same factors that induce expression of a regulated target. Alternatively, expression of the enzymatic nucleic acid molecule can be engineered to provide an additional level of regulation so as to limit the occurrence of the trans-splicing event to those conditions under which both the enzymatic nucleic acid molecule and target are selectively induced in the cell, but by different factors, the combination of those factors being the undesired event. Such regulation would allow the host cell to express the enzymatic nucleic acid molecule's target under those conditions in which the enzymatic nucleic acid molecule itself was not co-expressed.

The sequence of the trans-splicing enzymatic nucleic acid molecule domain that hybridizes to the target nucleic acid is determined by the sequence of the target nucleic acid. The sequence of the target nucleic acid is determined after cloning sequences encoding such nucleic acid, by sequencing a peptide encoded by such target and deducing an nucleic acid sequence that would encode such a peptide, or by using a database in which such sequence information is available, such as Genbank.

The selection of a desired sequence (the “trans-spliced sequence”) to be trans-spliced into the target nucleic acid sequence will reflect the purpose of the trans-splicing. If a trans-splicing event is desired that does not result in the expression of a new genetic sequence, then the trans-spliced sequence need not encode a translatable protein sequence. If a trans-splicing event is desired that does result in the expression of a new genetic sequence, and especially a new peptide, polypeptide, or protein sequence, then the trans-spliced sequence can further provide translational stop codons, and other information necessary for the correct translational processing of the nucleic acid in the host cell. If a specific protein product is desired as a result of the trans-splicing event, then preferably the amino acid reading frame is preserved in the resulting transcript.

The identification and confirmation of the specificity of a trans-splicing enzymatic nucleic acid molecule of the invention can be determined by testing the ability of a putative trans-splicing molecule to catalyze the desired trans-splicing reaction in the presence of the desired target sequence. The trans-splicing reaction should not occur if the only nucleic acid sequences present in the system are non-target sequences to which such trans-splicing enzymatic nucleic acid molecule should not be responsive (or less responsive). Such characterization can be performed with the assistance of a marker such that correct (or incorrect) trans-splicing enzymatic nucleic acid molecule activity can be more easily monitored. In most cases, it is sufficient to test the trans-splicing enzymatic nucleic acid molecule against its intended target in vitro and then transform a host cell with it for study of its in vivo effects.

The trans-splicing reaction of the invention need not be complete to provide a new genetic sequence to a host cell. It is an advantage of the invention that, depending upon the biological activity of the peptide that is translated from such genetic sequence, the trans-splicing event can in fact be quite inefficient, as long as sufficient trans-splicing occurs to provide sufficient mRNA and thus encoded polypeptide to the host for the desired purpose.

In one embodiment, transcription of the trans-splicing enzymatic nucleic acid molecule of the invention in a host cell occurs after introduction of a gene encoding trans-splicing enzymatic nucleic acid into the host cell. If the endogenous expression the trans-splicing enzymatic nucleic acid molecule by the host cell is not desired, such trans-splicing enzymatic nucleic acid molecule can be chemically or enzymatically synthesized and provided to the host cell by various methods described herein. Alternatively, when endogenous expression the gene encoding the ribozyme is desired, such expression can be achieved by stably inserting at least one DNA copy of the trans-splicing enzymatic nucleic acid molecule into the host's chromosome, or by providing a DNA copy of the trans-splicing enzymatic nucleic acid molecule on a plasmid that is stably retained by the host cell.

In another embodiment, the trans-splicing enzymatic nucleic acid molecule of the invention is inserted into the host's chromosome as part of an expression cassette, such cassette providing transcriptional regulatory elements that will control the transcription of the trans-splicing enzymatic nucleic acid molecule in the host cell. Such elements can include, but not necessarily be limited to, a promoter element (pol I, II or III elements), a T3 or T7 promoter, an enhancer or UAS element, a transcriptional terminator signal, a polyadenylation signal or a pol III termination signal.

In yet another embodiment, expression of a trans-splicing enzymatic nucleic acid molecule whose coding sequence has been stably inserted into a host's chromosome is controlled by the promoter sequence that is operably linked to the trans-splicing enzymatic nucleic acid molecule coding sequences. The promoter that directs expression of the trans-splicing enzymatic nucleic acid molecule can be any promoter functional in the host cell, prokaryotic promoters being desired in prokaryotic cells and eukaryotic promoters in eukaryotic cells. A promoter can be composed of discrete modules that direct the transcriptional activation and/or repression of the promoter in the host cell. Such modules can be mixed and matched in the trans-splicing enzymatic nucleic acid molecule's promoter so as to provide for the proper expression of the trans-splicing enzymatic nucleic acid molecule in the host. A eukaryotic promoter can be any promoter functional in eukaryotic cells, and especially can be any of an RNA polymerase I, II or III specificity. If it is desired to express the trans-splicing enzymatic nucleic acid molecule in a wide variety of eukaryotic host cells, a promoter functional in most eukaryotic host cells can be selected, such as a rRNA or a tRNA promoter, or the promoter for a widely expressed mRNA such as the promoter for an actin gene, or a glycolytic gene. If it is desired to express the trans-splicing enzymatic nucleic acid molecule only in a certain cell or tissue type, a cell-specific (or tissue-specific) promoter elements functional only in that cell or tissue type can be selected. Alternatively, the ribozyme can be expressed from a T3 or T7 promoter system by also supplying the appropriate T3 or T7 RNA polymerase proteins or expression cassettes.

In one embodiment, the trans-splicing reaction of the invention is chemically the same whether it is performed in vitro or in vivo.

Target Sites

Targets useful for trans-splicing enzymatic nucleic acid molecules of the invention can be determined as disclosed in Draper et al., U.S. Pat. No. 6,159,692; Sullivan et al., U.S. Pat. No. 5,989,906; and McSwiggen et al., U.S. Pat. No. 5,525,468 taking into account the sequence specificity of trans-splicing enzymatic nucleic acid molecules and target nucleic acid sequence information available electronically via a database or via traditional methods of cloning and sequencing. Using this sequence information, trans-splicing enzymatic nucleic acid molecules are designed to specifically interact with a target nucleic acid sequence via complementary hybridization and reprogram the target nucleic acid sequence via a trans-splicing reaction. The sequences of target nucleic acid molecules to be reprogrammed by methods of the invention can be screened for optimal trans-splicing enzymatic nucleic acid target sites using a computer-folding algorithm. The trans-splicing enzymatic nucleic acid molecules can be individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding regions and the catalytic core or the exon region are eliminated from consideration. In addition, varying binding region lengths can be chosen to optimize activity. Therefore, trans-splicing enzymatic nucleic acid molecule binding/trans-splicing sites are identified and trans-splicing enzymatic nucleic acids are designed to anneal to various sites in the nucleic acid target to be reprogrammed. The trans-splicing enzymatic nucleic acid molecules can be expressed via vectors or expression cassettes or can otherwise be chemically synthesized as described herein.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small” generally refers to nucleic acid motifs less than about 100 nucleotides in length) can be used for exogenous delivery. The simple structure of these molecules increases the ability of the nucleic acid to invade targeted regions of target nucleic acid structure. Exemplary molecules of the instant invention are chemically synthesized, and others can similarly be synthesized.

DNA based oligonucleotides including certain trans-splicing enzymatic nucleic acid molecules are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the these oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for RNA based oligonucleotides including certain trans-splicing enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al, 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA·3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing, the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive enzymatic nucleic acid molecules or binding attenuated control (BAC) oligonucleotides are synthesized by substituting nucleotides that are essential for catalytic activity of the enzymatic nucleic acid molecule. Therefore, one or more nucleotide substitutions can be introduced in enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). The nucleic acid molecules are purified by gel electrophoresis using general methods or are purified by high pressure liquid chromatography (HPLC; See Wincott et al., Supra, the totality of which is hereby incorporated herein by reference) and are re-suspended in water.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency (see e.g., Eckstein et al, International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications that enhance their efficacy in cells, and removal of bases from nucleic acid molecules to shorten oligonucleotide synthesis times and reduce chemical requirements are desired. All these publications are hereby incorporated by reference herein.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. S No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such a nucleic acid is also generally more resistant to nucleases than an unmodified nucleic acid. Thus, in a cell and/or in vivo the activity may not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

In one embodiment, nucleic acid molecules of the invention include one or more G-clamp nucleotides. A G-clamp nucleotide is a modified cytosine analog wherein modifications result in the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substation within an oligonucleotide can result in substantially enhanced helical thermal stability and mismatch discrimination when hybridized to complementary oligonucleotides. The inclusion of such nucleotides in nucleic acid molecules of the invention can enable both enhanced affinity and specificity to nucleic acid targets.

Therapeutic nucleic acid molecules delivered exogenously are optimally stable within cells until trans-splicing of the target nucleic acid occurs. This period of time varies between hours to days depending upon the disease state. Nucleic acid molecules that are delivered exogenously should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In another embodiment, the invention features conjugates and/or complexes of nucleic acid molecules of the invention. Compositions and conjugates are used to facilitate delivery of molecules into a biological system, such as cells. The conjugates provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel agents for the delivery of molecules, including but not limited to small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus based linkage, for example a phosphoramidite or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus containing group and saturated or unsaturated alkyl group, optionally substituted with OH, COOH, oxo, amine, or substituted or unsubstituted aryl groups.

In another embodiment, nucleic acid catalysts having chemical modifications that maintain or enhance enzymatic activity are provided. Such nucleic acids are also generally more resistant to nucleases than unmodified nucleic acid. Thus, in a cell and/or in vivo the activity of the nucleic acid may not be significantly lowered. As exemplified herein such enzymatic nucleic acids are useful in a cell and/or in vivo even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In non-limiting examples, the 3′-cap includes 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound that can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. The alkyl group can have, for example, 1 to 12 carbons. In one embodiment of the invention, the alkyl group is a lower alkyl of from 1 to 7 carbons. In another embodiment the alkyl group is 1 to 4 carbons. The alkyl group can be substituted or unsubstituted. When substituted the substituted group(s) can be hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. The alkenyl group can have, for example, 1 to 12 carbons. In one embodiment of the invention the alkenyl group can be a lower alkenyl of from 1 to 7 carbons. In another embodiment the alkenyl group can be 1 to 4 carbons. The alkenyl group can be substituted or unsubstituted. When substituted the substituted group(s) can be, for example, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. The alkynyl group can have, for example, 1 to 12 carbons. In one embodiment of the invention, the alkynyl group is a lower alkynyl of from 1 to 7 carbons. In another embodiment of the invention, the alkynyl group is 1 to 4 carbons. The alkynyl group can be substituted or unsubstituted. When substituted the substituted group(s) can be, for example, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base that contains a modification in the chemical structure of an unmodified nucleotide base, sugar and/or phosphate.

In connection with 2′-modified nucleotides as described for the present invention, by “amino” is meant 2′-NH₂ or 2′-O-NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of the nucleic acid-based molecules of the invention can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules. The treatment of patients with nucleic acid molecules can also include combinations of different types of nucleic acid molecules. Therapies can be devised which include a mixture of enzymatic nucleic acid molecules (including different enzymatic nucleic acid molecule motifs), antisense, siRNA and/or 2-5A chimera molecules to one or more targets to alleviate symptoms of a disease.

Administration of Nucleic Acid Molecules

The nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al., 2000, Cent. Nerv. Syst. Dis., 485-508; Peel and Klein, 2000, J. Neurosci. Methods, 98, 95-104; Hagihara et al, 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, RNA molecules of the present invention can be expressed from transcription units (see for example Couture et al., 1996, TIG., 12, 510) inserted into DNA or RNA vectors. The recombinant vectors can be DNA plasmids or viral vectors.

Ribozyme expressing viral vectors can be constructed based on, but not limited to, adeno-associated virus, retrovirus, adenovirus, or alphavirus. The recombinant vectors capable of expressing the nucleic acid molecules can be delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed.

The nucleic acid sequence encoding the nucleic acid molecule of the instant invention is operable linked in a manner that allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region in a manner that allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al, 1992, Proc. Natl. Acad. Sci. USA, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J, 11, 4411-8; Lisziewicz et al., 1993, Proc. Natl. Acad. Sci. U.S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein). The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising a nucleic acid sequence encoding at least one of the nucleic acid molecules of the invention in a manner that allows expression of that nucleic acid molecule. The expression vector comprises in one embodiment; a) a transcription initiation region; b) a transcription termination region; and c) a nucleic acid sequence encoding at least one said nucleic acid molecule, wherein said sequence is operably linked to said initiation region and said termination region in a manner that allows expression and/or delivery of said nucleic acid molecule.

In another embodiment the expression vector comprises: a) a transcription initiation region; b) a transcription termination region; c) an open reading frame; and d) a nucleic acid sequence encoding at least one said nucleic acid molecule, wherein said sequence is operably linked to the 3′-end of said open reading frame and wherein said sequence is operably linked to said initiation region, said open reading frame and said termination region in a manner that allows expression and/or delivery of said nucleic acid molecule. In yet another embodiment the expression vector comprises: a) a transcription initiation region; b) a transcription termination region; c) an intron; and d) a nucleic acid sequence encoding at least one said nucleic acid molecule, wherein said sequence is operably linked to said initiation region, said intron and said termination region in a manner which allows expression and/or delivery of said nucleic acid molecule.

In another embodiment, the expression vector comprises: a) a transcription initiation region; b) a transcription termination region; c) an intron; d) an open reading frame; and e) a nucleic acid sequence encoding at least one said nucleic acid molecule, wherein said sequence is operably linked to the 3′-end of said open reading frame and wherein said sequence is operably linked to said initiation region, said intron, said open reading frame and said termination region in a manner which allows expression and/or delivery of said nucleic acid molecule.

Alternately, nucleic acid molecules of the invention are administered exogenously to a patient, or to cells of a patient or other source that are later introduced to the patient. Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic nucleic acid molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. The nucleic acid molecules or the invention are administered via pulmonary delivery, such as by inhalation of an aerosol or spray dried formulation administered by an inhalation device or nebulizer. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (alleviate a symptom to some extent, or all of the symptoms) of a disease state in a patient.

The nucleic acid molecules of the invention can be administered and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., local administration or systemic administration, into a cell or patient, including, for example, a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “local administration” is meant in vivo local absorption or accumulation of drugs in the specific tissue, organ, or compartment of the body. Administration routes that can lead to local absorption include, without limitations: inhalation, direct injection, or dermatological applications.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired compound, e.g., nucleic acids, to an accessible diseased tissue. The rate of entry of a drug into the circulation has been shown to be a function of molecular weight or size. The use of a liposome or other drug carrier comprising the compounds of the instant invention, for example PEG or phospholipids conjugates, can potentially localize the drug, for example, in certain tissue types, such as the tissues of the reticular endothelial system (RES). A nucleic acid formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells.

Both local and systemic administration approaches can be used to administer nucleic acid molecules of the invention for the treatment of asthma or related conditions. In one embodiment, the nucleic acid molecule or formulation comprising the nucleic acid molecule is administered to a patient with an inhaler or nebulizer, providing rapid local uptake of the nucleic acid molecules into relevant pulmonary tissues. In another embodiment, the nucleic acid molecule or formulation comprising the nucleic acid molecule is administered to a patient systemically, for example by intravenous or subcutaneous injection, providing sustained uptake of the nucleic acid molecules into relevant bodily tissues.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al, International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, or all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

### EXAMPLES

The following are non-limiting examples showing the selection, isolation, synthesis and activity of nucleic acids of the instant invention.

### Example 1

### Identification of Potential Target Sites in a Target Nucleic Acid Sequence to be Reprogrammed

The sequence of target genes or target gene transcripts are screened for accessible sites using a computer-folding algorithm. Regions of the sequence that do not form secondary folding structures and contained potential enzymatic nucleic acid molecule trans-splicing sites are identified.

Accessible target sites are also identified in cell culture using a trans-splicing enzymatic nucleic acid molecule containing a randomized internal guide sequence and a spliced reporter domain. The enzymatic nucleic acid molecule can be supplied either as a synthetic RNA transcribed in vitro and transfected into a the appropriate cell type, or as part of an expression vector, either plasmid or viral, and produced intracellularly. Following an appropriate amount of time to allow trans-splicing to occur, such as 0.5-8 hours, total RNA is extracted from the transfected cells, and splicing products are amplified by PCR using a sense primer specific to the 5′ untranslated region of the target RNA, and an antisense primer specific to the reporter portion of the trans-splicing enzymatic nucleic acid molecule incorporated into the target RNA. The amplification product is then cloned and sequenced to determine the insertion sites, and the frequency of insertion sites quantified to identify the most accessible sites.

### Example 2

### Selection of Trans-splicing Enzymatic Nucleic Acid Molecules

Trans-splicing enzymatic nucleic acid molecule target sites are chosen by analyzing sequences of target nucleic acid sequences (eg. human, viral, bacterial etc.) and prioritizing the sites on the basis of folding. Trans-splicing enzymatic nucleic acid molecules are designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the guide region and targeted sequences and other regions of the molecule are generally eliminated from consideration. As noted below, targeting sequence region lengths can be varied to optimize activity.

### Example 3

### Expression Regulation via Trans-splicing, Reprogramming a Meal-Regulated Gene to Produce Insulin for the Treatment of Type I Diabetes

Type I late-stage type II diabetics produce inadequate amounts of insulin and thus are unable to regulate blood glucose levels. Normally, insulin production is tightly regulated to food intake. Currently, the disease is managed via daily insulin injections. However, in the absence of tight food intake-responsive regulation, current insulin replacement therapies eventually lead to serious complications such as kidney disease, nerve damage and amputations, blindness, heart disease and stroke. Therefore, there is a significant unmet need to provide insulin to such patients in a food intake-regulated manner.

Many genes in addition to insulin are regulated by food intake. Examples include the S14 gene in liver, the glucose-dependent insulinotropic polypeptide in intestinal K cells, and prominin in skeletal muscle. In this example, the coding region of the pro-insulin cDNA is cloned into a group I intron-derived trans-splicing enzymatic nucleic acid molecule that is targeted to the AUG initiation codon of any of the aforementioned genes responsive to food intake. The enzymatic nucleic acid molecule is cloned into an appropriate vector such as a plasmid, and placed downstream of a constitutive promoter such as the CMV promoter. The trans-splicing enzymatic nucleic acid molecule expression plasmid is complexed with cationic delivery vehicles to facilitate cellular uptake and administered either to muscle via intramuscular injection, to the liver via intravenous injection or via the hepatic artery, or to intestinal K cells via oral consumption of a time-release capsule containing the plasmid/lipid vehicle complexes. The trans-splicing enzymatic nucleic acid molecule is constitutively expressed in the target tissue, but insulin is not produced until the target RNA regulated by food intake is expressed and trans-splicing occurs. Thus, insulin production is regulated based on availability of the food intake—regulated target mRNA substrate.

### Example 4

### Trans-splicing Mediated Cancer-Specific Expression, Reprogramming of a Cancer-Specific Gene for Cancer-specific Chemotherapy, Immunotherapy and Marking

Cancer cells are genetically unstable and exhibit a variety of abnormal properties in addition to uncontrolled cell growth, such as expression of fetal genes or other genes not normally expressed in somatic cells. Often, such genes can serve as markers to distinguish cancer cells from normal cells. Examples of genes expressed specifically in cancer cells include, but are not limited to, the carcinoembryonic antigen (CEA) gene in colorectal carcinoma; prostate cancer specific antigen (PSA) and breast cancer antigen MUC-1, the melanoma-associated antigens MART-100 and gp100, and the MAGE, SAGE, GAGE and LAGE/NY-ESO.1 gene families.

Cancer-specific chemotherapy. Chemotherapeutic agents are effective anticancer agents because they kill actively dividing cancer cells. Unfortunately, such agents also kill normal cells that that are actively dividing, such as cells lining the gut and hematopoictic cells, leading to toxic side effects. One strategy to circumvent such side effects is to express exogenous enzymes in cancer cells that convert harmless pro-drugs into active chemotherapeutic agents. This strategy has been referred to as “suicide gene therapy” because the transferred gene leads to death of its host cell. Examples of such exogenous enzymes are the herpes simplex thymidine kinase, the cytosine deaminase gene, the varicellazoster virus thymidine kinase gene and the E. coli Deo gene. The difficulty with such suicide gene therapy strategies is delivering the exogenous gene specifically to cancer cells in order to avoid killing normal cells. In this example, trans-splicing enzymatic nucleic acid molecules are used to circumvent the delivery problem by reprogramming only cancer-specific genes. One of the aforementioned exogenous enzymes, such as herpes thymidine kinase (HSV-tk), is cloned into a group I intron-derived trans-splicing enzymatic nucleic acid molecule that is targeted to the AUG initiation codon of any of the aforementioned cancer-specific genes. The enzymatic nucleic acid molecule is cloned into an appropriate viral vector, such as an adenoviral vector, adeno-associated viral vector, or a retroviral vector, and placed downstream of a constitutive promoter such as the CMV promoter, or the LTR promoter of the retroviral vector. The trans-splicing enzymatic nucleic acid molecule expression vector is administered either locally via intratumoral injection, or systemically via intravenous administration. The trans-splicing enzymatic nucleic acid molecule is constitutively expressed in all cell types and tissues transduced by the viral vectors, but the exogenous suicide enzyme is selectively produced only in cancer cells expressing the cancer-specific target RNA and after trans-splicing occurs. A pro-drug such as gancyclovir is administered to the patient. The pro-drug is converted to the active chemotherapeutic agent by the exogenous HSV-tk suicide enzyme only in the cancer cells, leading to cancer-specific killing.

Cancer immunotherapy. Immunostimulating agents are introduced into cancer cells in order to make the cancer cells targets for destruction by the immune system. Examples of immunostimulatory agents used in such cancer immunotherapy approaches are HLA-B7,G-CSF, GM-CSF, Interferons and Interleukins. One challenge in reducing such immunotherapy strategies to practice is accomplishing cancer-specific expression, especially when attempting to target distant metastases via systemic administration, thereby avoiding immune responses to normal tissues. In this example, the coding region for one of the aforementioned antigenic proteins such as HLA-B7 is cloned into a group I intron-derived trans-splicing enzymatic nucleic acid molecule that is targeted to the AUG initiation codon of any of the aforementioned cancer-specific genes. The enzymatic nucleic acid molecule is cloned into an appropriate viral vector such as an adenoviral vector, adeno-associated viral vector, or a retroviral vector, and placed downstream of a constitutive promoter such as the CMV promoter or the LTR promoter of the retroviral vector. The trans-splicing enzymatic nucleic acid molecule expression vector is administered either locally via intratumoral injection, or systemically via intravenous administration. The trans-splicing enzymatic nucleic acid molecule is constitutively expressed in all cell types and tissues transduced by the viral vectors, but the exogenous immunomodulator is selectively produced only in cancer cells expressing the cancer-specific target RNA and after trans-splicing occurs. In this manner, only the cancer cells expressing the cancer-specific target RNA produce the antigen and become targets for immune destruction.

Cancer marking. Surgical removal of diffuse tumors such as brain tumors and certain tumors of epithelial origin can be challenging because the tumor borders are difficult to identify. In this example, tumor borders are detected by trans-splicing a reporter gene into a cancer-specific mRNA and detecting such cancer cells via expression of the reporter. Examples of reporters include fluorescent proteins such as green fluorescent protein which can be detected via laser induced fluorescence spectroscopy, and the beta-galactosidase protein encoded by the bacterial lacZ gene which can be detected using the colormetric substrate 5-bromo-4-chloro-3-indolyl-beta-D-galacto-(arentyougladyoutookorganic)-pyranoside (X-gal). The coding region for one of the aforementioned reporter proteins is cloned into a group I intron-derived trans-splicing enzymatic nucleic acid molecule that is targeted to the AUG initiation codon of any of the aforementioned cancer-specific genes. The enzymatic nucleic acid molecule is cloned into an appropriate viral vector such as an adenoviral vector, adeno-associated viral vector, or a retroviral vector, and placed downstream of a constitutive promoter such as the CMV promoter or the LTR promoter of the retroviral vector. The trans-splicing enzymatic nucleic acid molecule expression vector is administered either locally via intratumoral injection, or systemically via intravenous administration. The trans-splicing enzymatic nucleic acid molecule is constitutively expressed in all cell types and tissues transduced by the viral vectors, but the marker protein is selectively produced only in cancer cells expressing the cancer-specific target RNA and after trans-splicing occurs. In this manner, only the cancer cells expressing the cancer-specific target RNA are marked, enabling accurate detection of the tumor borders.

### Example 5

### Localized Production of a Growth Factor, Reprogramming Muscle-specific Genes to Produce VEGF for the Treatment of Peripheral Vascular Disease

Peripheral Vascular Disease occurs as a result of arteriosclerosis, or hardening of the arteries, due to plaque formation. This results in inadequate blood flow to tissues causing ischemia which leads to intense pain at rest and often requires limb amputation. In this example, trans-splicing enzymatic nucleic acid molecules are used to reprogram muscle-specific mRNAs to produce angiogenic proteins to recruit growth of new blood vessels to ischemic tissues. Examples of angiogenic proteins include the VEGF family and the FGF family of growth factors. The coding region of one of the aforementioned angiogenic proteins such as VEGF is cloned into a group I intron-derived trans-splicing enzymatic nucleic acid molecule that is targeted to the AUG initiation codon of any of the mRNAs from any of the numerous skeletal muscle-specific genes such as the MADS superfamily of transcription factors. In this example, the trans-splicing enzymatic nucleic acid molecule is produced synthetically via the in vitro T7 system by placing the T7 promoter upstream of the trans-splicing enzymatic nucleic acid molecule, then transcribing in vitro and purifying said enzymatic nucleic acid molecule using methods well known in the art. The trans-splicing enzymatic nucleic acid molecule is complexed with cationic delivery vehicles to facilitate cellular uptake and administered to the affected area via intra-muscular injection. VEGF is produced only when the trans-splicing enzymatic nucleic acid molecule has entered muscle cells and trans-splicing has occurred with the muscle-specific mRNA. VEGF is not produced if the enzymatic nucleic acid molecule enters a non-muscle cell, thereby controlling sites of angiogenesis. The VEGF locally produced will recruit new blood vessel formation/growth in the treated areas, thus alleviating the ischemic condition.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The terms and expressions that have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Examples of Biopharmaceuticals

Name
Epidermal Growth Factor (EGF)
Endocrine Gland Derived Vascular Endothelial Growth Factor
(EG-VEGF)
Erythropoietin
Fibroblast Growth Factor Family (FGF acidic/basic, FGF4-10, FGF16-18)
FMS-like tyrosine kinase 3 ligand (FLT3-Ligand)
Granulocyte-Colony Stimulating Factor (G-CSF)
Granulocyte Macrophage Colony Stimulating Factor (GM-C SF)
Human Growth hormone (HGH)
Insulin
Interferon leukocyte
Interleuken-1 alpha (IL-1 alpha)
Interleuken-1 beta (IL-1 Beta)
Interleuken-2 (IL-2)
Interleuken-3 (IL-3)
Interleuken-4 (IL-4)
Interleuken-5 (IL-5)
Interleuken-6 (TL-6)
Interleuken-7 (TL-7)
Interleuken-8 (IL-8) (72 a.a. form or 77 a.a. form)
Interleuken-10 (IL-10)
Interleuken-11 (IL-11)
Interleuken-12 (IL-12)
Interleuken-13 (IL-13)
Interleuken-15 (IL-15)
Interleuken-16 (IL-16)
Lnterleuken-16 (IL-16, 121aa)
Interleuken-17 (IL-17)
Interleuken-18 (IL-18)
Interleuken-19 (IL-19)
Interleuken-20 (IL-20)
Interferon-inducible protein 10 (IP-10)
Leptin
Macrophage-Colony Stimulating Factor (M-CSF)
Nerve Growth Factor Beta (NGF-Beta)
Pleiotrophin
Transforming Growth Factor Alpha (TGF-Alpha)
Transforming Growth Factor Beta 1 (TGF-Beta 1)
Transforming Growth Factor Beta 2 (TGF-Beta 2)
Transforming Growth Factor Beta 3 (TGF-Beta 3)
Tumor Necrosis Factor Alpha (TNF-Alpha)
Tumor Necrosis Factor Beta (TNF-Beta)
Tumor Necrosis Factor receptor type I (sTNF-receptor I)
Tumor Necrosis Factor receptor type II (sTNF-receptor II)
Vascular Endothelial Growth Factor (VEGF)

TABLE II

| | | | | | |
|---|---|---|---|---|---|
| | | | | Wait Time* | Wait Time* |
| Reagent | Equivalents | Amount | Wait Time* DNA | 2′-O-methyl | RNA |

A. 2.5 μmol Synthesis Cycle ABI 394 Instrument

| | | | | | | | | | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| | | | | | | | | | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | Equivalents: DNA/ | Amount: DNA/ | | | | Wait Time* | | Wait Time* | |
|---|---|---|---|---|---|---|---|---|---|
| Reagent | 2′-O-methyl/Ribo | 2′-O-methyl/Ribo | | Wait Time* DNA | | 2′-O-methyl | | Ribo | |
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

What we claim is:

1. An enzymatic nucleic acid molecule having trans-splicing activity, wherein the enzymatic nucleic acid molecule comprises sequence encoding a biopharmaceutical and comprises sequence complementary to a target nucleic acid.

2. The enzymatic nucleic acid molecule of claim 1, wherein said sequence encoding a biopharmaceutical comprises RNA.

3. The enzymatic nucleic acid molecule of claim 1, wherein said sequence encoding a biopharmaceutical comprises DNA.

4. The enzymatic nucleic acid molecule of claim 1, wherein said biopharmaceutical is expressed in vitro.

5. The enzymatic nucleic acid molecule of claim 1, wherein said biopharmaceutical is expressed in vivo.

6. The enzymatic nucleic acid molecule of claim 1, wherein said biopharmaceutical comprises compounds shown in Table I.

7. A method comprising:
a. providing the enzymatic nucleic acid molecule of claim 1 and a substrate comprising a predetermined target RNA for the enzymatic nucleic acid molecule in vitro under reaction conditions that promote trans-splicing activity of the enzymatic nucleic acid molecule; and
b. reacting the enzymatic nucleic acid molecule with the substrate.

8. A method comprising:
a. providing the enzymatic nucleic acid molecule of claim 1 and a substrate comprising a predetermined target DNA for the enzymatic nucleic acid molecule in vitro under reaction conditions that promote trans-splicing activity of the enzymatic nucleic acid molecule; and
b. reacting the enzymatic nucleic acid molecule with the substrate.

9. A method comprising introducing into a cell under conditions suitable for trans-splicing activity, the enzymatic nucleic acid molecule of claim 1, wherein the enzymatic nucleic acid molecule further comprises binding arms complementary to a target RNA sequence in the cell, and wherein said enzymatic nucleic acid molecule introduces the sequence encoding the biopharmaceutical into the target RNA sequence such that said biopharmaceutical is expressed under the genetic control of said host cell.

10. The method of claim 9, wherein said target RNA molecule is a messenger RNA (mRNA).

11. The method of claim 9, wherein said target RNA molecule is a pre-messenger RNA (pre-mRNA).

12. The method of claim 9, wherein said target RNA molecule is a viral RNA.

13. A method comprising introducing into a cell under conditions suitable for trans-splicing activity, the enzymatic nucleic acid molecule of claim 1, wherein the enzymatic nucleic acid molecule further comprises binding arms complementary to a target DNA sequence in the cell, and wherein said enzymatic nucleic acid molecule introduces the sequence encoding the biopharmaceutical into said target DNA sequence such that said biopharmaceutical is expressed under the genetic control of said host cell.

14. The method of claim 13, wherein said target DNA is chromosomal DNA.

15. The method of claim 13, wherein said target DNA is viral DNA.

16. A RNA expression vector encoding the enzymatic nucleic acid molecule of claim 1.

17. A DNA expression vector encoding the enzymatic nucleic acid molecule of claim 1.

18. A RNA expression vector encoding the enzymatic nucleic acid molecule of claim 2.

19. A DNA expression vector encoding the enzymatic nucleic acid molecule of claim 3.

20. A method comprising:
a. introducing into a cell the expression vector of claim 16 under conditions suitable for the expression and trans-splicing activity of said enzymatic nucleic acid molecule in the cell; and
b. expressing the enzymatic nucleic acid molecule to reprogram the expression of a target RNA sequence in the cell by introducing a sequence encoding a biopharmaceutical into the target RNA sequence under conditions suitable for the biopharmaceutical to be expressed in the cell.

21. The method of claim 20, wherein said target RNA molecule is a messenger RNA (mRNA), pre-messenger RNA (pre-mRNA), or viral RNA.

22. A method comprising:
a. introducing into a cell the expression vector of claim 17 under conditions suitable for the expression and trans-splicing activity of said enzymatic nucleic acid molecule in the cell; and
b. expressing the enzymatic nucleic acid molecule to reprogram the expression of a target DNA sequence in the cell by introducing a sequence encoding a biopharmaceutical into the target DNA sequence under conditions suitable for the biopharmaceutical to expressed in the cell.

23. The method of claim 22, wherein said target DNA molecule is chromosomal DNA or viral DNA.

24. A method for generating enzymatic nucleic acid molecules with trans-cleaving activity capable of biopharmaceutical production comprising:
a. generating a randomized pool of oligonucleotides, wherein a portion of each oligonucleotide comprises a fixed sequence that encodes a biopharmaceutical product and wherein a portion of each oligonucleotide comprises sequence complementary to a predetermined nucleic acid target sequence;
b. isolating sequences from said pool that possess trans-cleaving activity;
c. amplifying said sequences isolated from (b) under conditions suitable for introducing some degree of mutation into said sequences; and
d. repeating steps (b) and (c) under conditions suitable for isolating enzymatic nucleic acid molecules with trans-cleaving activity capable of introducing said sequence encoding a biopharmaceutical product into said nucleic acid target sequence.

25. The method of claim 24, wherein said predetermined nucleic acid target sequence is chromosomal DNA or viral DNA.

26. The method of claim 24, wherein said predetermined nucleic acid target sequence is a messenger RNA (mRNA), pre-messenger RNA (pre-mRNA), or viral RNA.

27. The enzymatic nucleic acid molecule of claim 1, wherein said enzymatic nucleic acid molecule comprises about 8 to about 100 bases complementary to a target nucleic acid.

28. The enzymatic nucleic acid molecule of claim 1, wherein said enzymatic nucleic acid molecule comprises about 14 to about 24 bases complementary to a target nucleic acid.

29. A mammalian cell comprising the enzymatic nucleic acid molecule of claim 1.

30. The mammalian cell of claim 29, wherein said cell is a human cell.

31. A method of expressing a biopharmaceutical composition in a cell comprising contacting said cell with the enzymatic nucleic acid molecule of claim 1, under conditions suitable for said expression.

32. A method of treating a patient having a disease, illness, or condition that can be treated with a biopharmaceutical compound comprising contacting cells of the patient with the enzymatic nucleic acid molecule of claim 1 under conditions suitable for said treatment.

33. The method of claim 32, wherein said method further comprises the use of one or more drug therapies under conditions suitable for said treatment.

34. A method of trans-splicing a sequence encoding a biopharmaceutical compound into a target RNA sequence comprising contacting the enzymatic nucleic acid molecule of claim 1 with the target RNA molecule under conditions suitable for trans-splicing the sequence encoding the biopharmaceutical compound into the target RNA.

35. A method of trans-splicing a sequence encoding a biopharmaceutical compound into an target DNA sequence comprising contacting the enzymatic nucleic acid molecule of claim 1 with the target DNA molecule under conditions suitable for trans-splicing the sequence encoding the biopharmaceutical compound into the target DNA.

36. The enzymatic nucleic acid molecule of claim 1, wherein the enzymatic nucleic acid molecule comprises a group I intron.

37. The enzymatic nucleic acid molecule of claim 1, wherein the enzymatic nucleic acid molecule comprises a group II intron.

38. The enzymatic nucleic acid molecule of claim 1, wherein the enzymatic nucleic acid molecule comprises a pre-messenger RNA intron.

39. An expression vector comprising a nucleic acid sequence encoding at least one enzymatic nucleic acid trans-splicing molecule of claim 1 in a manner which allows expression of the enzymatic nucleic acid molecule.

40. A mammalian cell comprising the expression vector of claim 39.

41. A method for treatment of a patient comprising administering to the patient the enzymatic nucleic acid molecule of claim 1 under conditions suitable for the treatment.

42. The method of claim 41 further comprising administering to the patient one or more other therapies.

43. A method of administering to a mammal the enzymatic nucleic acid molecule of claim 1 comprising contacting the mammal with the enzymatic nucleic acid trans-splicing molecule under conditions suitable for the administration.

44. The method of claim 43, wherein said mammal is a human.

45. The method of claim 43, wherein said administration is in the presence of a delivery reagent.

46. The method of claim 45, wherein said reagent comprises a lipid, cationic lipid, phospholipid, or liposome.

47. A method of administering to a mammal the expression vector of claim 39 comprising contacting the mammal with said vector under conditions suitable for said administration.

48. The method of claim 47, wherein said administration is in the presence of a delivery reagent.

49. The method of claim 48, wherein said reagent comprises a lipid, cationic lipid, phospholipid, or liposome.

50. A method of administering to a patient the enzymatic nucleic acid molecule of claim 1 in conjunction with a therapeutic agent comprising contacting said patient with said enzymatic nucleic acid molecule and said therapeutic agent under conditions suitable for said administration.

51. A composition comprising the enzymatic nucleic acid molecule of claim 1 and a pharmaceutically acceptable carrier.
